(19) United States
(12) Patent Application Publication
(10) Pub. No.: US 2004/0198974 A1
(43) Pub. Date: Oct. 7, 2004

(54) Chromatographic separation of enantiomers of protected amino acids via smb-method

(76) Inventors: Kurt Guenther, Erlensee (DE); Stefan Merget, Rodgau (DE); Karlheinz Drauz, Freigericht (DE); Hans-Peter Krimmer, Dietzenbach (DE); Michael Schwarm, Alzenau (DE)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/486,200

(22) PCT Filed: Jul. 3, 2002
(86) PCT No.: PCT/EP02/07345

Related U.S. Application Data
(60) Provisional application No. 60/312,747, filed on Aug. 17, 2001.

(51) Int. Cl. C07D 267/22; C07D 281/18; C07D 291/00; C07D 337/16; C07D 487/00; C07D 498/00; C07D 513/00

(52) U.S. Cl. 540/456

(57) ABSTRACT

The present invention is directed to the separation of enantiomers of racemates of formula (I). The separation proceeds by applying deemed racemates to continuos enantioselective chromatography like SMB. The methods predominantly is performed for industrial scale production of pure enantiomers of deemed amino acids which are useful intermediates in organic synthesis.

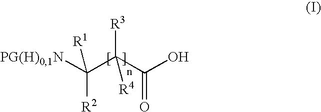

The instant invention is concerned with the separation of enantiomers of racemic compounds of formula (I).

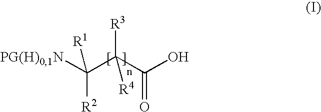

Especially the invention deals with a chromatographic method called SMB (simulated moving bed).

Enantiomerically enriched compounds of present formula (I) are important intermediates for production of bioactives in organic synthesis.

There are numerous strategies to produce instant compounds enantioselectively e.g. by way of synthesis, enzymatically or via classical separation of racemates.

However, it is still an objective to find further possibilities for their production, since not all known methods yield all of the compounds of formula (I) in advantageous results especially with respect to their enantiomeric excess.

Therefore, the problem underlying the instant invention is to find other ways to generate highly enantiomerically enriched compounds of formula (I). Especially it is sought to create a process for the mentioned production which is advantageously applied in chemical industries on technical scale and serves to gain such compounds in an ecological and economical superior way.

This approach is successfully realized by utilization of a procedure for the production of enantiomerically enriched compounds of formula (I)

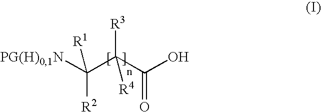

wherein

PG is a mono- or bidentate protective group for amino functions

n is 0, 1, 2

R¹, R² independently of each other represent H, (C₁-C₁₂)-alkyl, (C₂-C₈)-alkenyl, (C₂-C₈)-alkynyl, (C₁-C₈)-alkoxy, (C₁-C₈)-alkoxyalkyl, (C₃-C₈)-cycloalkyl, (C₆-C₁₈)-aryl, (C₇-C₁₉)-aralkyl, (C₃-C₁₈)-heteroaryl, (C₄-C₁₉)-heteroaralkyl, ((C₁-C₈)-alkyl)₁₋₃-(C₃-C₈)-cycloalkyl, ((C₁-C₈)-alkyl)₁₋₃-(C₆-C₁₈)-aryl, ((C₁-C₈)-alkyl)₁₋₃-(C₃-C₁₈)-heteroaryl or the two radicals are bonded to one another via a (C₁-C₈)-alkylene bridge,

R³, R⁴ independently of each other and independently with respect to different n represent H, (C₁-C₈)-alkyl, (C₂-C₈)-alkenyl, (C₂-C₈)-alkynyl, (C₁-C₈)-alkoxy, (C₁-C₈)-alkoxyalkyl, (C₃-C₈)-cycloalkyl, (C₆-C₁₈)-aryl, (C₇-C₁₉)-aralkyl, (C₃-C₁₈)-heteroaryl, (C₄-C₁₉)-heteroaralkyl, ((C₁-C₈)-alkyl)₁₋₃-(C₃-C₈)-cycloalkyl, ((C₁-C₈)-alkyl)₁₋₃-(C₆-C₁₈)-aryl, ((C₁-C₈)-alkyl)₁₋₃-(C₃-C₁₈)-heteroaryl or the two radicals are bonded to one another via a (C₁-C₈)-alkylene bridge

or R¹ and R³ are bonded to one another via a (C₁-C₈)-alkylene bridge,

by separating the racemate of chiral compounds of formula (I) on chiral phases by means of liquid SMB-chromatography. Racemates of the general formula (I) can readily be converted into the desired enantiomerically enriched protected amino acids by aid of the known SMB-chromatography, whereby a novel approach to obtaining that class of compounds has been opened up.

Preference is given to compounds of the general formula (I) wherein the protective group PG is removable by acidic or basic hydrolysis or hydrogenolysis, such as selected from the group comprising Z, Fmoc, Boc, phthaloyl, acetyl, Moc, Eoc, Alloc, formyl, propionyl, butyryl, isobutyryl, benzoyl, carbamoyl, propoxycarbonyl, butoxycarbonyl, isopropoxycarbonyl, wherein the aromatic rings of these groups can optionally be substituted by one or more heteroatomic residues like F, Cl, Br, I, OH, MeO, EtO, PrO, BuO, tBuO, Pho, NO₂, CF₃.

Also preferred are compounds of the general formula (I) in which n is O, R¹, R² independently of each other represent H, (C₁-C₁₂)-alkyl, (C₃-C₈)-cycloalkyl, (C₆-C₁₈)-aryl, (C₃-C₁₈)-heteroaryl or the two radicals are bonded to one another via a (C₁-C₈)-alkylene bridge.

Also preferred are compounds of the general formula (I) in which n is O, R¹, R² independently of each other represent H, methyl, ethyl, propyl, butyl, isopropyl, 2-butyl, tert-butyl, adamantyl, neopentyl, cyclohexyl, methyl thioethyl, 1-hydroxyethyl, propagyl, cyclopentyl. Utmostly preferred is the compound Z-tert-leucine.

The SMB-chromatography is a method for a continuos liquid chromatography known to the artisan and perfectly applicable for separation problems on industrial scale (Mazzotti et al. Chiral Europe 1996, 103f.; Strube et al. Organic Process Research & Development 1998, 2, 305-319; Juza et al. GIT Spezial Chromatographie 1998, 2, 108f.; EP0878222; Schulte et al. Chemie Ingenieur, Technik 1966, 68, 670-683).

SMB-method according to the invention is preferably performed with chiral phases selected from the group comprising silicagels impregnated with sugar derivatives or micro-crystalline esters of cellulose. Also preferred is a procedure according to the invention wherein the chiral phases are silicagels impregnated with amylose derivatives. Phases like these are commercially availyble e.g. Chiralpak AS® or OD® from Daicel.

The skilled worker is free to use a solvent or solvent mixture as mobile phase appropriate for the invention. Preferably mobile phases selected from the group comprising water, acetonitril, alcohols, like methanol or ethanol, alcanes, like hexane, isohexane, organic acids, like acetic acid, formic acid, TFA are used.

The temperature during separation should be adapted to the procedure to secure the most efficient preparative effect. Preference is given to a procedure wherein the temperature during chromatography lies between 10° C. and 40° C., preferably between 20° C. and 30° C. Most preferably the temperature is around 25° C.

Also the flow rates of the mobile phase can be regulated according to the skilled workers mind. Preferably in the procedure according to the invention the flow rate is within the range of 0.2-2 ml/min, preferably 0.8-1.2 ml/min, most preferably around 1 ml/min.

The pressure of the mobile phase can be adjusted according to the best separation results. Predominantly, the procedure according to the invention is run with a pressure within the range of 20-50 bar, preferably 30-40 bar, most preferably 35 bar.

Operational issues not addressed in the above may be adapted like known in the art or can for purposes of increasing the separation efficiency be arranged according to the skilled workers knowledge.

(C₁-C₈)-Alkyl may be regarded as being methyl, ethyl, n-propyl, isopropyl, n-butyl, isobutyl, sec-butyl, tert-butyl, pentyl, hexyl, heptyl or octyl including all isomers due to different positions of the double bond. They may be mono- or poly-substituted or by (C₁-C₈)-alkoxy, (C₁-C₈)-haloalkyl, OH, halogen, NH₂, NO₂, SH, S—(C₁-C₈)-alkyl. (C₁-C₁₂)-alkyl may be a (C₁-C₈)-alkyl residue with 4-atoms in excess. The alkyl residue may optionally be substituted or may contain within its chain one or more of the heteroatoms of the group O, S, Se, Cl, F, Br, I, N, P, Si, Ge.

(C₂-C₈)-alkenyl is to be understood as being a (C₁-C₈)-alkyl radical as described above, with the exception of methyl, that has at least one double bond.

(C₂-C₈)-alkynyl is to be understood as being a (C₁-C₈)-alkyl radical as described above, with the exception of methyl, that has at least one triple bond.

(C₃-C₈)-cycloalkyl is to be understood as being cyclopropyl, cyclobutyl, cyclopentyl, cyclohexyl and cycloheptyl radicals, etc.. They may be substituted by one or more halogens and/or radicals containing an N, O, P, S atom and/or may have in the ring radicals containing an N, O, P, S atom, such as, for example, 1-, 2-, 3-, 4-piperidyl, 1-, 2-, 3-pyrrolidinyl, 2-, 3-tetrahydrofuryl, 2-, 3-, 4-morpholinyl. They may also be mono- or poly-substituted by (C₁-C₈)-alkoxy, (C₁-C₈)-haloalkyl, OH, Cl, NH₂, NO₂.

A (C₆-C₁₈)-aryl radical is to be understood as being an aromatic radical having from 6 to 18 carbon atoms. Such radicals include especially compounds such as phenyl, naphthyl, anthryl, phenanthryl, biphenyl radicals. It may be mono- or poly-substituted by (C₁-C₈)-alkoxy, (C₁-C₈)-haloalkyl, OH, halogen, NH₂, NO₂, SH, S—(C₁-C₈)-alkyl.

A (C₇-C₁₉)-aralkyl radical is a (C₆-C₁₈) -aryl radical bonded to the molecule via a (C₁-C₈)-alkyl radical.

(C₁-C₈)-alkoxy is a (C₁-C₈)-alkyl radical bonded to the molecule in question via an oxygen atom.

(C₁-C₈)-haloalkyl is a (C₁-C₈)-alkyl radical substituted by one or more halogen atoms.

Within the scope of the invention, a (C₃-C₁₈)-heteroaryl radical denotes a five-, six- or seven-membered aromatic ring system of from 3 to 18 carbon atoms that contains hetero atoms such as, for example, nitrogen, oxygen or sulfur in the ring. Such heteroaromatic radicals are to be regarded as being especially radicals such as 1-, 2-, 3-furyl, such as 1-, 2-, 3-pyrrolyl, 1-, 2-, 3-thienyl, 2-, 3-, 4-pyridyl, 2-, 3-, 4-, 5-, 6-, 7-indolyl, 3-, 4-, 5-pyrazolyl, 2-, 4-, 5-imidazolyl, acridinyl, quinolinyl, phenanthridinyl, 2-, 4-, 5-, 6-pyrimidinyl. It may be mono- or poly-substituted by (C₁-C₈)-alkoxy, (C₁-C₈)-haloalkyl, OH, halogen, NH₂, NO₂, SH, S—(C₁-C₈)-alkyl.

A (C₄-C₁₉)-heteroaralkyl is to be understood as being a heteroaromatic system corresponding to the (C₇-C₁₉)-aralkyl radical.

The expression (C₁-C₈)-alkylene unit is to be understood as meaning a (C₁-C₈)-alkyl radical that is bonded to the molecule in question via two single bonds of its carbon atoms. It may be mono- or poly-substituted by (C₁-C₈)-alkoxy, (C₁-C₈)-haloalkyl, OH, halogen, NH₂, NO₂, SH, S—(C₁-C₈) -alkyl.

Suitable halogens are fluorine, chlorine, bromine and iodine.

Within the scope of the invention, the expression enantiomerically concentrated is to be understood as meaning the proportion of an enantiomer in admixture with its optical antipodes in a range >50% and <100%.

### EXAMPLE

The following is an example of a computational calculation of a simulated moving bed separation of Z-tert.-leucine.

Method B: CHIRALPAK® AD

Analytic injection:

Column: CHIRALPAK®AD 20 μm 250*4.6 mm

Mobile Phase: ACN+0.1% TFA

Flow Rate: 1 ml/min

Temperature: 25° C.

Detection: DAD 275 nm

| Concentration | Injection | Load | Tr (1) | Tr (2) | | | | | | |
|---|---|---|---|---|---|---|---|---|---|---|
| (g/l) | Volume (ml) | (mg) | (min) | (min) | K′ (1) | K′ (2) | N (1) | N(2) | α | Rs |
| 1 | 0.02 | 0.02 | 4.00 | 5.36 | 0.33 | 0.79 | 1619 | 1024 | 2.36 | 2.55 |

Loading Data:

Separation Conditions:

Column: CHIRALPAK®AD 20 μm 250*4.6 mm

Mobile Phase: ACN+0.1% TFA

Flow Rate: 1 ml/min

Temperature: 25° C.

Detection: DAD 275 nm

| Concen- | Injection | | | | | | | |
|---|---|---|---|---|---|---|---|---|
| tration | Volume | Load | tr (1) | tr (2) | | | | |
| (g/l | (ml) | (mg) | (min) | (min) | k′ (1) | k′ (2) | N (1) | N (2) |
| 300.14 | 0.01 | 3.0014 | 3.79 | 4.69 | 0.26 | 0.56 | 1025 | 362 |
| 300.14 | 0.02 | 6.0028 | 3.71 | 4.53 | 0.24 | 0.51 | 804 | 272 |
| 300.14 | 0.03 | 9.0042 | 3.65 | 4.45 | 0.22 | 0.48 | 725 | 220 |
| 300.14 | 0.04 | 12.0056 | 3.57 | 4.40 | 0.19 | 0.47 | 794 | 171 |
| 300.14 | 0.05 | 15.007 | 3.55 | 4.35 | 0.18 | 0.45 | 986 | 140 |
| 300.14 | 0.08 | 24.0112 | 3.47 | 4.19 | 0.16 | 0.40 | 1391 | — |

Simulation Results:

Isotherm Parameters:

| | |
|---|---|
| λ | 0.6 |
| 1. NK1 | 0.37 |
| NK2 | 0.83 |
| Nbar | 34 |
| Function | 0.044 |
| Porosity | 0.391 |
| Reliability of | low |
| isotherm | |

1.1.

1.2. SMB Parameter Estimation

(a) For Licosep 8-50

SMB Operating Pressure=35 bar.

| | | |
|---|---|---|
| 8 col. (g of CSP) | 800 | 800 |
| Feed Flow (ml/min) | 16.46 | 37.03 |
| Feed Concentration (g/l) | 160.00 | 54.00 |
| Recycle Flow Rate (ml/min) | 365.83 | 354.62 |
| Extract Flow Rate (ml/min) | 116.15 | 110.10 |
| Raffinate Flow Rate (ml/min) | 30.59 | 45.04 |
| Switch Time (period) (min) | 0.70 | 0.73 |
| Zone I flow (=recycle) (ml/min) | 365.83 | 354.62 |
| Zone II flow (ml/min) | 249.68 | 244.52 |
| Zone III flow (ml/min) | 266.14 | 281.55 |
| Zone IV flow (ml/min) | 235.55 | 236.51 |
| Average Flow Rate (ml/min) | 279.3 | 279.3 |
| Extract Purity (% ee) | 99.66 | 99.36 |
| Extract Concentration (g/L) | 11.34 | 9.08 |
| Raffinate Purity (% ee) | 99.9 | 99.9 |
| Raffinate Concentration (g/l) | 43.05 | 22.20 |
| Production Rate g/day enantiomer | 1896.59 | 1439.85 |
| Solvent consumption (1/day) | 211.32 | 223.40 |
| Productivity (g enantiomer/kg/day) | 2370.73 | 1799.81 |

(b) For Production Scale Operation

Feed concentration: 160 g/l

| Column | Recycle | | Extract | Raffinate | Production |
|---|---|---|---|---|---|
| Diameter | Flow | Feed Flow | Flow | Flow | Rate |
| (cm) | (l/hr) | (l/hr) | (l/hr) | (l/hr) | (MTA) |
| 20 | 351 | 16 | 112 | 29 | 10.0 |
| 40 | 1405 | 63 | 446 | 117 | 40.1 |
| 60 | 3161 | 142 | 1004 | 264 | 90.1 |
| 80 | 5619 | 253 | 1784 | 470 | 160.2 |
| 100 | 8780 | 395 | 2788 | 734 | 250.3 |

Feed concentration: 54 g/l

| Column | Recycle | | Extract | Raffinate | Production |
|---|---|---|---|---|---|
| Diameter | Flow | Feed Flow | Flow | Flow | Rate |
| (cm) | (l/hr) | (l/hr) | (l/hr) | (l/hr) | (MTA) |
| 20 | 340 | 36 | 106 | 43 | 7.6 |
| 40 | 1362 | 142 | 423 | 173 | 30.4 |
| 60 | 3064 | 320 | 951 | 389 | 68.4 |
| 80 | 5447 | 569 | 1691 | 692 | 121.6 |
| 100 | 8511 | 889 | 2642 | 1081 | 190.1 |

These results can be obtained by using a mathematical estimation program of Chiral Technologies Europe. It is believed that real live conditions will lead to approximately the same results.

1. Procedure for the production of enantiomerically enriched compounds of formula (I) 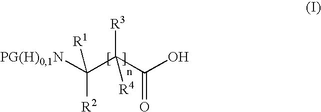
wherein
PG is a mono- or bidentate protective group for amino functions
n is 0,1,2
R¹, R² independently of each other represent H, (C₁-C₁₂)-alkyl, (C₂-C₈)-alkenyl, (C₂-C₈)-alkynyl, (C₁-C₈)-alkoxy, (C₁-C₈)-alkoxyalkyl, (C₃-C₈)-cycloalkyl, (C₆-C₁₈)-aryl, (C₇-C₁₉)-aralkyl, (C₃-C₁₈)-heteroaryl, (C₄-C₁₉)-heteroaralkyl, ((C₁-C₈)-alkyl)₁₋₃-(C₃-C₈)-cycloalkyl, ((C₁ C₈)-alkyl)₁₋₃-(C₆-C₁₈)-aryl, ((C₁-C₈)-alkyl)₁₋₃-(C₃-C₁₈)-heteroaryl or the two radicals are bonded to one another via a (C₁-C₈)-alkylene bridge,
R³, R⁴ independently of each other and independently with respect to different n represent H, (C₁-C₈)-alkyl, (C₂-C₈)-alkenyl, (C₂-C₈)-alkynyl, (C₁-C₈)-alkoxy, (C₁-C₈)-alkoxyalkyl, (C₃-C₈)-cycloalkyl, (C₆-C₁₈)-aryl, (C₇-C₁₉)-aralkyl, (C₃-C₁₈)-heteroaryl, (C₄-C₁₉)-heteroaralkyl, ((C₁-C₈)-alkyl)₁₋₃-(C₃-C₈)-cycloalkyl, ((C₁-C₈)-alkyl)₁₋₃-(C₆-C₁₈)-aryl, ((C₁-C₈)-alkyl)₁₋₃-(C₃-C₁₈)-heteroaryl or the two radicals are bonded to one another via a (C₁-C₈)-alkylene bridge
or R¹ and R³ are bonded to one another via a (C₁-C₈)-alkylene bridge,
by separating the racemate of chiral compounds of formula (I) on chiral phases by means of liquid SMB-chromatography.

2. Procedure according to claim 1 wherein the protective group PG is removable by acidic or basic hydrolysis or hydrogenolysis, such as selected from the group comprising Z, Fmoc, Boc, phthaloyl, acetyl, , Moc, Eoc, Alloc, formyl, propionyl, butyryl, isobutyryl, benzoyl, carbamoyl, propoxycarbonyl, butoxycarbonyl, isopropoxycarbonyl, wherein the aromatic rings can optionally be substituted by one or more heteroatomic residues like F, Cl, Br, I, OH, MeO, EtO, PrO, BuO, tBuO, Pho, NO₂, CF₃.

3. Procedure according to claim 1 wherein
n is 0
R¹, R² independently of each other represent H, (C₁-C₁₂)-alkyl, (C₃-C₈)-cycloalkyl, (C₆-C₁₈)-aryl, (C₃-C₁₉)-heteroaryl or the two radicals are bonded to one another via a (C₁-C₈)-alkylene bridge.

4. Procedure according to claim 1 wherein
n is 0
R¹, R² independently of each other represent H, methyl, ethyl, propyl, butyl, isopropyl, 2-butyl, , tert-butyl, adamantyl, neopentyl, cyclohexyl, methyl thioethyl, 1-hydroxyethyl, propagyl, cyclopentyl.

5. Procedure according to claim 1 wherein the chiral phases are selected from the group comprising silicagels impregnated with sugar derivatives or micro-crystalline esters of cellulose.

6. Procedure according to claim 1 wherein the chiral phases are silicagels impregnated with amylose derivatives.

7. Procedure according to claim 1 wherein the mobile phase is selected from the group comprising water, acetonitril, alcohols, like methanol or ethanol, alcanes, like hexane, isohexane, organic acids, like acetic acid, formic acid, TFA.

8. Procedure according to claim 1 wherein the temperature during chromatography lies between 10° C. and 40° C., preferably between 20° C. and 30° C.

9. Procedure according to claim 1 wherein the flow rate is within the range of 0.2-2 ml/min, preferably 0.8-1.2 ml/min.

10. Procedure according to claim 1 wherein the pressure is kept within the range of 20-50 bar, preferably 30-40 bar.
